(19) United States
(12) Patent Application Publication
(10) Pub. No.: US 2010/0286794 A1
(43) Pub. Date: Nov. 11, 2010

(54) ENDOPROSTHESIS FOR ANATOMICAL DUCT

(75) Inventor: Bruno Ferreyrol, La Ciotat (FR)

Correspondence Address: J. Rodman Steele;Novak Druce & Quigg LLP, 525 Okeechobee Blvd, Suite 1500, West Palm Beach, FL, 33401, US

(73) Assignee: NOVATECH SA, LA CIOTAT (FR)

(21) Appl. No.: 12/811,942

(22) PCT Filed: Jan. 5, 2009
(86) PCT No.: PCT/FR09/00002

§ 371 Date: Jul. 7, 2010

(30) Foreign Application Priority Data
Jan. 10, 2008 (FR) 0800139

(51) Int. Cl. A61F 2/82

(52) U.S. Cl. 623/23.64

(57) ABSTRACT

According to the invention, the one-piece tubular body (2) of the endoprosthesis comprises a flexible intermediate part (21) permitting the relative orientation of the parts (2A, 2B) of said body (2) that are situated on either side of said flexible intermediate part (21).

The present invention is directed to a one-piece endoprosthesis for an anatomical duct. For example, from American U.S. Pat. No. 5,236,446, there is already known an endoprosthesis for an anatomical duct made of a generally cylindrical, one-piece tubular body, defininig an internal lumen over its whole length. Such an endoprosthesis is mainly intended for maintaining opened said anatomical duct, when the latter tends to become clogged or to collapse. As a result, said endoprosthesis should exhibit a high crushing resistance, so that it is made of a biocompatible semi-rigid material, such as a silicone elastomer.

Using such an endoprosthesis does not result in any difficulty when the location of the anatomical duct where it is positioned has the shape of said prosthesis. However, difficulties occur as soon as the shape of said location is curved or when the shape of said location is likely to be modified after the endoprosthesis has been positioned.

Then, the only known solution is to manufacture, at high cost, such an endoprosthesis on a tailor-made way.

Such a drawback of shape dissimilarity between the anatomical duct and the endoprosthesis is all the worse because it has been recently found that the above described endoprosthesis could be successfully used for internally reinforcing a tubular graft intended for providing the junction between sections of an anatomical duct, part of which has been removed. Indeed, when a part of an anatomical duct such as, for example, the trachea or a bronchus, is the seat of a malignant tumor, a surgery could be implemented—the protocol of which has been established by Professor Emmanuel Martinod comprising the steps of removing said part of the anatomical duct and replacing it with a tubular graft, for instance, an aorta or artery part, providing the junction between sections of said anatomical duct arranged on both sides of said removed part. Said endoprosthesis reinforces the junction between said sections of the anatomical duct and prevents said graft from collapsing.

However, it may occur that said endoprosthesis cannot have the right wanted anatomical conformance and/or that said sections of the anatomical duct move one with respect to the other after the graft has been positioned.

The object of the present invention thus aims at improving the known above described endoprosthesis, so as to allow it to adapt at least partially to the anatomical conformance and/or the relative mobility of the anatomical duct, it is inserted into.

To this end, according to the invention, the endoprosthesis for internally supporting a tubular graft providing the junction between two sections of an anatomical duct, said endoprosthesis comprising a tubular body made of a biocompatible semi-rigid material and defining an internal lumen over its whole length, is remarkable in that said tubular body comprises at least one flexible tubular intermediate part being integral with said tubular body, so that the assembly of it and said flexible tubular intermediate part is integral, and allowing for the relative orientation of the semi-rigid parts of said one-piece tubular body located on both sides of said flexible intermediate part.

Thus, according to the present invention, said endoprosthesis, while being crush resistant due to the semi-rigid parts thereof, has properties for adapting to the conformance and to the deformation of said anatomical duct.

Preferably, said intermediate part is flexible because it is externally ringed.

As a function of the shape of said anatomical duct, said one-piece tubular body could have a cylindrical, conical, bifurcated general shape, etc.

When said one-piece tubular body has a bifurcated shape, and consequently, said endoprosthesis is adapted for an anatomical duct being itself bifurcated, it comprises at least:
one common tubular part;
two bifurcated tubular parts; and
at least one flexible intermediate part, arranged between said common tubular part and one of said bifurcated tubular parts.

So it is possible to vary the angle between the two bifurcated tubular parts in order to adapt the endoprosthesis according to the invention for the anatomical angle of said bifurcated duct.

Advantageously, each end of the one-piece tubular body is provided with an external overthickness ring. Thus, by means of said thick end rings, the ends of the endoprosthesis according to the invention are rigid enough as to efficiently anchor said endoprosthesis in the anatomical duct.

Preferably, in order to facilitate the anchoring of said rings in said anatomical duct, the latter comprise external side projections. Such anchoring projections can be consists in studs, such as those described in EP 1,681,040. In such a case, said projections could, in addition to the anchoring function thereof, act as a reservoir for active product or as a housing for a radio-opaque product or a label.

Furthermore, it is known that some anatomical ducts do not have an homogeneous mechanical resistance at the periphery thereof. In such a case, advantageously, the endoprosthesis according to the invention is more resistant than the weakest part of the anatomical duct. To this end, at least a peripheral portion of at least one part of said one-piece body, other than said flexible intermediate part, has projecting external longitudinal ribs, allowing for said peripheral portion to be stiffened.

It should be noticed that the recess formed between two adjacent projecting longitudinal ribs could act as a housing for an active product or similar as a layer or a sheet.

The figures of the appended drawing will make it easier to understand how this invention could be implemented. In these figures, similar reference numerals relate to similar components.

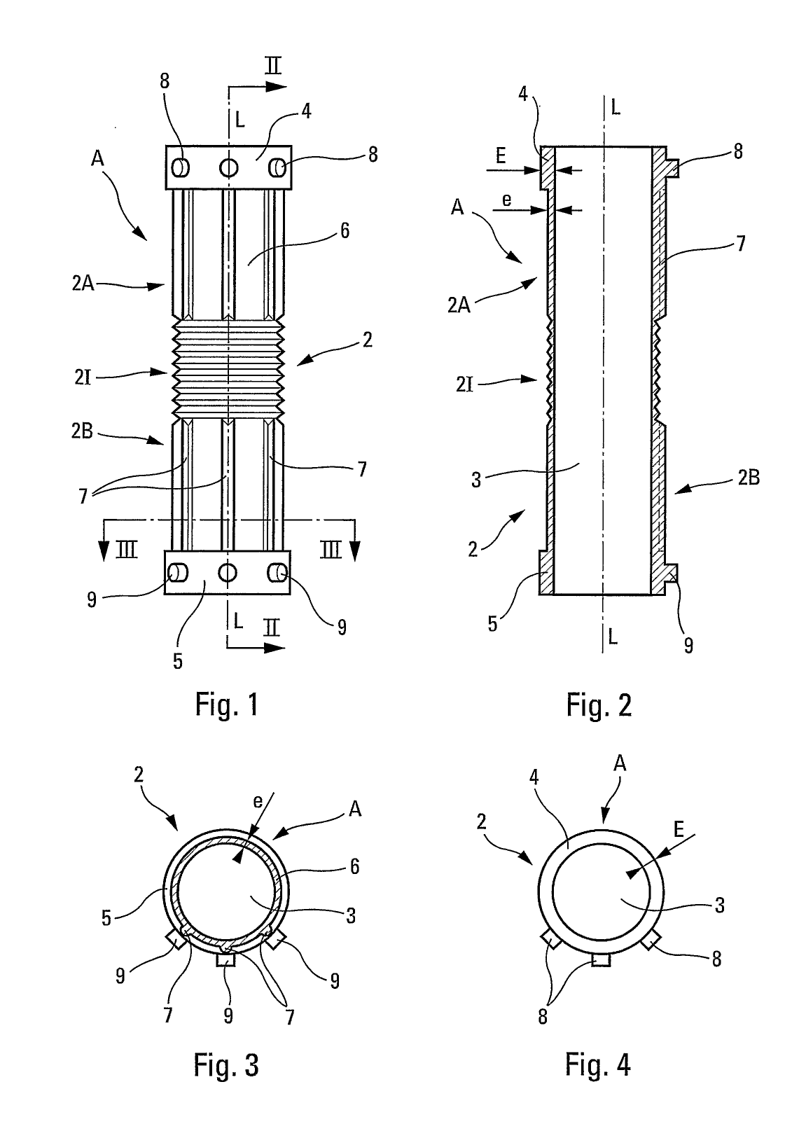

FIG. 1 depicts an exemplary embodiment of the one-piece prosthesis according to the present invention.

FIGS. 2 and 3 are respectively an axial section and a cross section of the prosthesis on FIG. 1, according to lines II-II and III-III of the latter.

FIG. 4 is a top view of the prosthesis of FIG. 1.

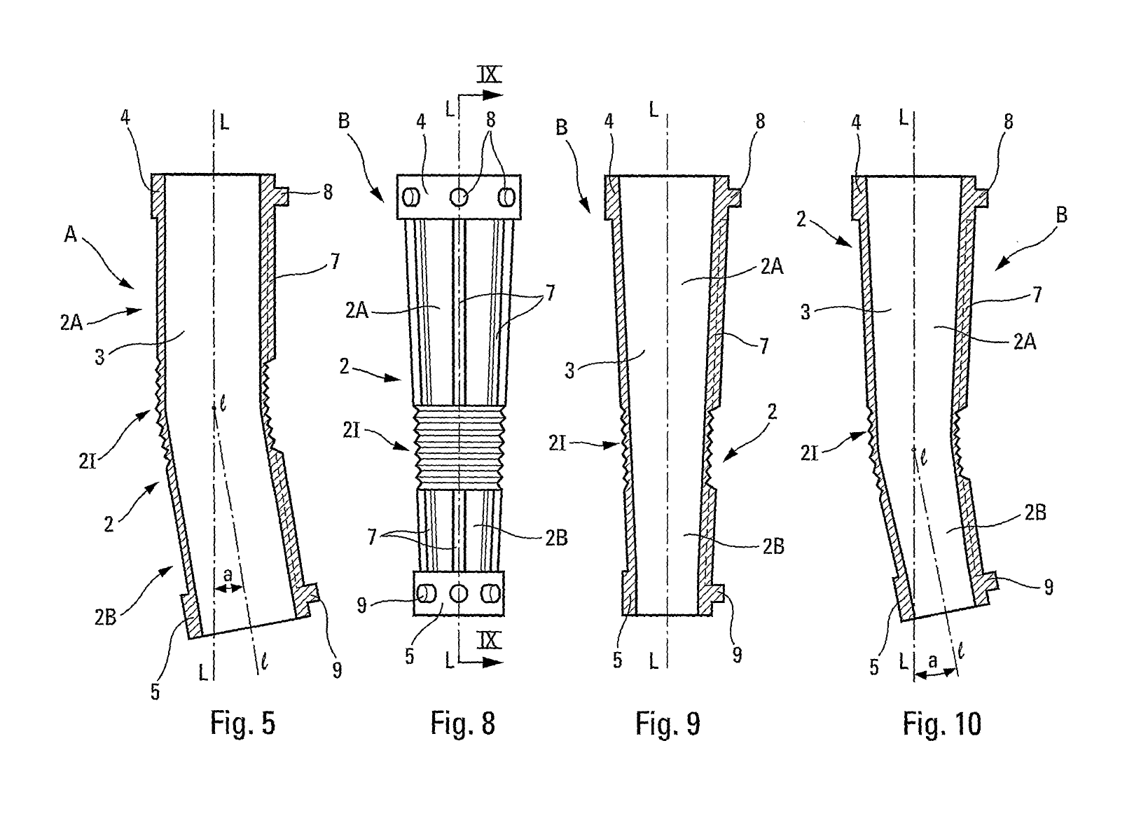

FIG. 5 illustrates, on a view similar to that of FIG. 2, the orientation possibility of an end of the prosthesis with respect to the other.

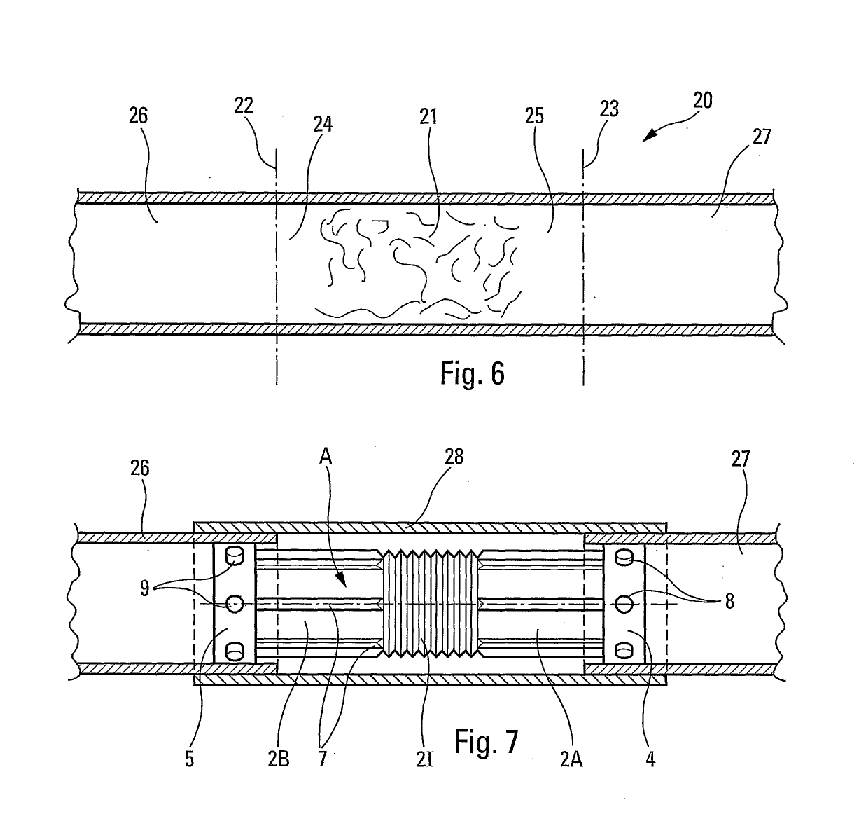

FIGS. 6 and 7 illustrate an embodiment of the prosthesis according to the invention.

FIG. 8 depicts an embodiment of the one-piece prosthesis according to the present invention.

FIG. 9 corresponds to a longitudinal section of the prosthesis of FIG. 8, along the line IX-IX.

FIG. 10 illustrates, on a view similar to that of FIG. 9, the orientation possibility of an end of the prosthesis with respect to the other.

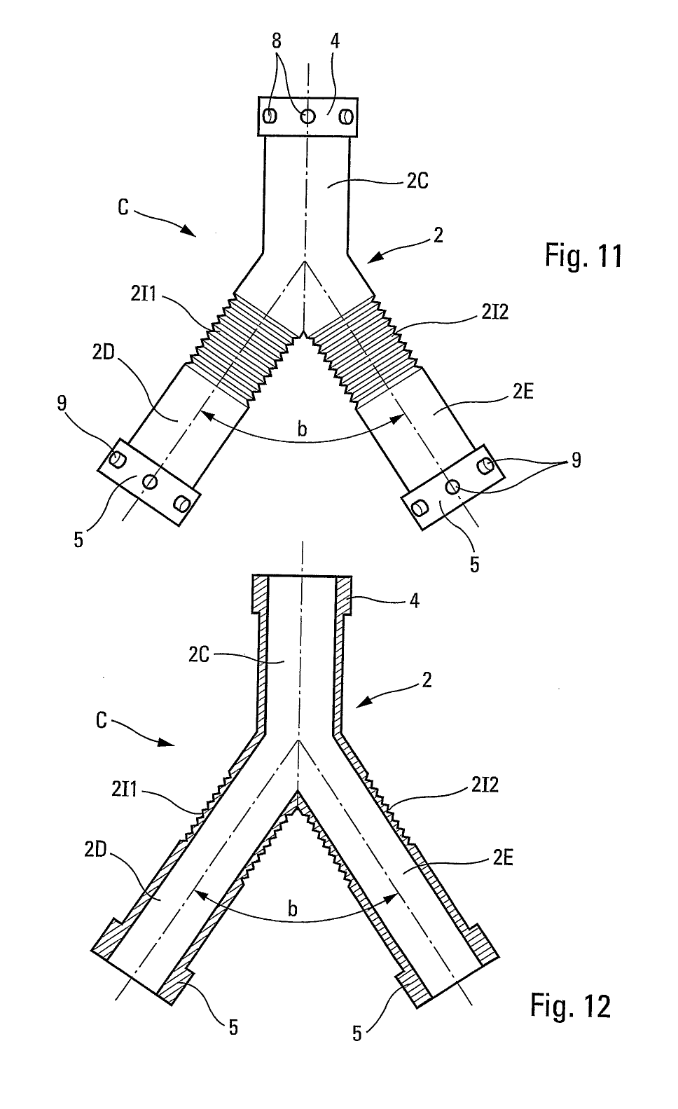

FIG. 11 depicts another examplary embodiment of the one-piece prosthesis according to the present invention.

FIG. 12 is a longitudinal section of the prosthesis of FIG. 12.

The prosthesis A according to the present invention and illustrated by FIGS. 1 to 5, is made of a one-piece tubular body 2, for example, made of a semi-rigid silicone elastomer, defining a smooth and uniform internal lumen 3, with a longitudinal axis L-L. The one-piece tubular body 2 comprises an intermediate tubular part 21, externally ringed so as to be flexible, connecting two semi-rigid tubular parts 2A and 2B.

As illustrated on FIG. 5, such an intermediate part 21 imparts some transversal flexibility to said body 2, so that the two parts 2A and 2B thereof are able to be oriented in a limited way one with respect to the other. On this figure, it has been shown that the axis L-L of the part 2B could be deflected by an angle a with respect to the axis L-L of the prosthesis and of the part 2A.

Furthermore, the semi-rigid tubular parts 2A and 23 are respectively provided with end rings 4, 5 projecting with respect to the tubular wall 6 of said parts 2A and 23. Indeed, the thickness e of the tubular wall 6 is smaller than the thickness E of the rings 4 and 5, thus projecting to the exterior with respect to said tubular wall 6. The latter comprises external longitudinal stiffening ribs 7.

Said stiffening ribs 7 could be evenly distributed around the tubular wall 6; in contrast, as shown on the figures, they could be only arranged on part of the periphery of said tubular wall 6 of the walls 2A and 2B.

The rings 4 and 5 support externally projecting studs 8 and 9, respectively, for instance, similar to those described in EP 1,681,040.

The above described endoprosthesis A can be used for internally reinforcing an anatomical duct tending to collapse or for maintaining the lumen of an anatomical duct tending to become clogged.

As illustrated on FIGS. 6 and 7, the endoprosthesis A could also be implemented for supporting a graft intended to connect two sections of an anatomical duct.

On FIG. 6, there is shown, in a partial schematic view, an anatomical duct 20, having a part 21 being the seat of a malignant tumor. Said part 21 is removed from the anatomical duct 20 through resection along cutting lines 22, 23 arranged in the healthy parts 24 and 25 of the latter, located on both sides of said tumor. After said part 21 and the healthy parts 24 and 25 have been resected, the anatomical duct 20 thus comprises two separated sections 26, 27.

As is illustrated on FIG. 7, the two separated sections 26, 27 are then connected by the prosthesis A, the rings 4, 5 and the studs 8, 9 being housed inside said sections, whereas a graft 28 connects them externally, surrounding said prosthesis A.

It is readily understood that, by means of the flexible intermediate part 21 thereof, the prosthesis A could adapt to a possible relative shift of one of both sections 26, 27 with respect to the other, after the latter and the graft 28 become integral with each other.

In the examplary embodiment A shown on FIGS. 1 to 5 and 7, the body 2 and the lumen 3 have an at least approximately cylindrical general shape.

Alternatively, in the examplary embodiment B on FIGS. 8 to 10, the body 2 and the lumen 3 have an at least approximately conical general shape. Thus, the one-piece endoprosthesis B could adapt to an also conical anatomical duct. Through its flexible intermediate part 21, said endoprosthesis B has a high adaptation capacity to said anatomical duct (see FIG. 10).

The embodiment C of the one-piece endoprosthesis according to the present invention, shown on FIGS. 11 and 12, is intended for a bifurcated anatomical duct and has itself a bifurcated shape. The one-piece body 2 of such endoprosthesis C comprises:
a common tubular part 2C, comparable to the part 2A as described above;
two bifurcated tubular parts 2D and 2E, comparable to the part 2B as described above and
two flexible intermediate parts 211 and 212, comparable to the part 21 described above and respectively arranged between said common tubular part 2C and one of said bifurcated tubular parts 2D, 2E.

From the herein above description, it is easily understood that the preferably ringed flexible intermediate parts 211 and 212 allow the angle b between the branches 2D and 2E of the bifurcation to adapt to the anatomy of the bifurcated anatomical duct for which it is provided.

1. An endoprosthesis for internally supporting a tubular graft providing the junction between two sections of an anatomical duct, said endoprosthesis comprising a tubular body (2) made in a biocompatible semi-rigid material and defining an internal lumen (3) over its whole length,
wherein said tubular body (2) comprises at least one flexible tubular intermediate part (21; 211, 212) being integrally part of said tubular body (2), so that the assembly of it and said flexible tubular intermediate part is one piece, and allowing for the relative orientation of the semi-rigid parts of said one-piece tubular body (2) located on both sides of said flexible intermediate part.

2. An endoprosthesis according to claim 1,
wherein said flexible intermediate part (21; 211, 212) is externally ringed.

3. An endoprosthesis according to claim 1, wherein said one-piece tubular body (2) has a cylindrical general shape.

4. An endoprosthesis according to claim 1, wherein said one-piece tubular body (2) has a conical general shape.

5. An endoprosthesis according to claim 1, wherein said one-piece tubular body (2) has a bifurcated shape.

6. An endoprosthesis according to claim 5,
wherein said bifurcated tubular body (2) comprises:
one common tubular part;
two bifurcated tubular parts (2D, 2E); and
at least one flexible intermediate part (211, 212), arranged between said common tubular part and one of said bifurcated tubular parts.

7. An endoprosthesis according to claim 1, wherein each end of said one-piece tubular body (2) is provided with a ring (4, 5) with an external overthickness.

8. An endoprosthesis according to claim 7,
wherein said end rings (4, 5) comprise external side projections (8, 9).

9. An endoprosthesis according to claim 8,
wherein said external side projections (8, 9) have the shape of studs.

10. An endoprosthesis according to claim 1, wherein at least one part of said one-piece tubular body (2), other than said flexible intermediate part, has external longitudinal ribs (7).

11. An endoprosthesis according to claim 10,
wherein the spaces being defined between said external longitudinal ribs (7) contain an active product.

12. An endoprosthesis according to claim 10, wherein said external longitudinal ribs (7) are unevenly distributed at the periphery of said tubular parts.
